(19) United States
(12) Patent Application Publication
(10) Pub. No.: US 2024/0307671 A1
(43) Pub. Date: Sep. 19, 2024

(54) LYMPHANGIOGENESIS INDUCING DEVICE

(71) Applicant: TERUMO KABUSHIKI KAISHA, Tokyo (JP)

(72) Inventor: Manami Kawasaki, Kanagawa (JP)

(73) Assignee: TERUMO KABUSHIKI KAISHA, Tokyo (JP)

(21) Appl. No.: 18/671,024

(22) Filed: May 22, 2024

Related U.S. Application Data
(63) Continuation of application No. PCT/JP2022/044012, filed on Nov. 29, 2022.

(30) Foreign Application Priority Data
Nov. 30, 2021 (JP) 2021-193934

(51) Int. Cl. A61M 37/00

(52) U.S. Cl. CPC A61M 37/0069; A61M 2202/0007; A61M 2202/06; A61M 2205/04; A61M 2210/04

(57) ABSTRACT

A lymphangiogenesis inducing device includes: an outer needle including a needle point insertable into a subcutaneous tissue, and a through-hole extending in an axial direction and penetrating the outer needle; and an inner needle inserted into the through-hole. The inner needle includes a cavity portion extending along the axial direction, and a wound imparting structure that is formed in a part of a distal end configured to protrude past the needle point of the outer needle and configured to cause a wound on the subcutaneous tissue. The wound imparting structure includes one or more protrusions protruding outward from the inner needle.

### CROSS-REFERENCE TO RELATED APPLICATIONS

The is a bypass continuation of PCT/JP2022/044012, filed on Nov. 29, 2022, which claims priority to Japanese Application No. JP2021-193934, filed on Nov. 30, 2021. The entire contents of these applications are incorporated herein by references.

### BACKGROUND

The present disclosure relates to a lymphangiogenesis inducing device used in a procedure for inducing lymphangiogenesis.

A lymphatic vessel is one of routes for recovering tissue fluid in a living body. A blockage in a lymphatic vessel stagnates tissue fluid and may develop into lymphedema accompanied by functional deterioration such as swelling and sensory paralysis in the extremities, or the arms and legs. Lymphedema is often developed by lymphadenectomy or radiation therapy performed as part of cancer treatment such as breast cancer treatment.

Lymphedema is a disease that is difficult to eliminate completely once it has been developed. If lymphedema becomes chronic, it is difficult to ameliorate, and if lymphedema is neglected, the condition gets worse. As a treatment for such lymphedema, a method of implanting a filamentous substance in a subcutaneous tissue has been proposed (JP 2020-127607 A, WO 2020/189157 A, JP 2021-104161 A, and Brorson H. Liposuction Gives Complete Reduction of Chronic Large Arm Lymphedema After Breast Cancer. Acta Oncologica, 2000; 39 (3): 407-20.).

Furthermore, Joseph M R et al., Characterization of lymphangiogenesis in a model of adult skin regeneration. Am J Physiol Heart Circ Physiol. 2006 September; 291(3): H1402-H1410., and Tammela & Alitalo, Lymphangiogenesis: Molecular mechanisms and future promise. Cell. 2010 Feb. 19; 140(4): 460-76. report on mechanisms of lymphangiogenesis during typical wound healing. Joseph M R et al. report that macrophages accumulate in a wound due to an immune response to the wound, and the accumulated macrophages produce vascular endothelial growth factors C (VEGF-C), thereby inducing lymphangiogenesis. In addition, Tammela & Alitalo report that lymphangiogenesis originates and emerges from an existing lymphatic vessel.

### SUMMARY

According to devices disclosed in JP 2020-127607 A, WO 2020/189157 A, and JP 2021-104161 A, it has become clear that a certain effect on lymphangiogenesis can be obtained with a simple procedure. The procedure for inducing lymphangiogenesis desires a more highly effective lymphangiogenesis inducing device.

An object of certain embodiments of the the present disclosure is to solve the above-described problem.

Embodiments of a lymphangiogenesis inducing device according to the present disclosure are described below.

According to one embodiment, a lymphangiogenesis inducing device includes: an outer needle including a needle point insertable into a subcutaneous tissue and a through-hole extending in an axial direction and penetrating the outer needle; and an inner needle inserted into the through-hole, wherein the inner needle includes a cavity portion (for example, a lumen) extending along the axial direction and a wound imparting structure that is formed in a part of a distal end exposable from the needle point of the outer needle and causes a minor wound on the subcutaneous tissue, and the wound imparting structure includes at least one protrusion protruding outward from the inner needle.

According to the lymphangiogenesis inducing device, the wound imparting structure is used to cause a wound and to indwell an implant, thereby inducing lymphangiogenesis in the subcutaneous tissue. With this configuration, the lymphangiogenesis inducing device induces lymphangiogenesis by growth factors of immune cells during wound healing and induces lymphangiogenesis by various cells fixed to the implant. Accordingly, the lymphangiogenesis inducing device exerts a great effect in inducing lymphangiogenesis.

According to one aspect of the lymphangiogenesis inducing device, the inner needle may include a plurality of protrusions protruding radially outward from a diameter of a proximal end of the inner needle. According to this lymphangiogenesis inducing device, it is possible to cause many minor wounds on the subcutaneous tissue by the plurality of protrusions.

According to one aspect of the lymphangiogenesis inducing device, the protrusions may have a folding mechanism that is folded when inserted into the through-hole and unfolded outward when protruding from the needle point of the outer needle. According to this lymphangiogenesis inducing device, the protrusions are unfolded outward so that it is possible to give damage to a radially larger area of the subcutaneous tissue than the through-hole. Since a large area of the subcutaneous tissue is wounded, this lymphangiogenesis inducing device exerts an excellent lymphangiogenesis effect.

According to one aspect of the lymphangiogenesis inducing device, the inner needle may have a proximal end provided with an inner needle hub, and the inner needle may be capable of protruding or retracting from the needle point of the outer needle by operation of the inner needle hub. This lymphangiogenesis inducing device facilitates the operation of the inner needle and has excellent operability.

According to one aspect of the lymphangiogenesis inducing device, the inner needle hub may include a hollow portion communicating with the cavity portion of the inner needle and may allow a drug solution or an implant to be supplied to the cavity portion of the inner needle through the inner needle hub. This lymphangiogenesis inducing device can facilitate indwelling of the implant and injection of the drug solution and has excellent operability.

According to one aspect of the lymphangiogenesis inducing device, the implant may be a porous and filamentous biodegradable polymer. Such an implant can further promote lymphangiogenesis.

According to one aspect of the lymphangiogenesis inducing device, the implant may be indwelled in the subcutaneous tissue after the outer needle and the inner needle are drawn out of the subcutaneous tissue. When a cell that induces lymphangiogenesis is fixed to this implant, it is possible to promote lymphangiogenesis.

According to one aspect of the lymphangiogenesis inducing device, the lymphangiogenesis inducing device may further include an implant placed in advance in the cavity portion of the inner needle. This lymphangiogenesis inducing device enables an operator to save time and effort for inserting the implant into the inner needle and reduces the burden on the operator during use.

According to certain embodiments of the lymphangiogenesis inducing device, it is possible to promote lymphangiogenesis during healing of wounds formed on the subcutaneous tissue by the protrusions. Furthermore, it is possible to indwell the implant effective for lymphangiogenesis in the subcutaneous tissue through the cavity portion of the inner needle, which further enhances the lymphangiogenesis effect together with the wound healing effect.

### BRIEF DESCRIPTION OF THE DRAWINGS

FIG. 1 is a schematic cross-sectional view of a lymphangiogenesis inducing device according to a first embodiment.

FIG. 2 is a cross-sectional view perpendicular to the axial direction of an outer needle and an inner needle taken along line II-II in FIG. 1.

FIG. 3A is a schematic cross-sectional view illustrating a state where a long implant is inserted into a lumen of the inner needle illustrated in FIG. 1, and FIG. 3B is a schematic cross-sectional view illustrating a state where a syringe containing a drug solution is connected to an inner needle hub illustrated in FIG. 1.

FIG. 4 is a flowchart illustrating a lymphangiogenesis method using the lymphangiogenesis inducing device illustrated in FIG. 1.

FIG. 5A is a view for describing a step of puncturing a target area with the outer needle and the inner needle, and FIG. 5B is a view for describing a step of placing an implant in the lumen of the inner needle.

FIG. 6A is a view for describing a step of drawing the outer needle out of the target area, and FIG. 6B is a view for describing a step of drawing the inner needle out of the target area while leaving the implant in the target area and a step of cutting off the implant.

FIG. 7A is a view for describing a step of puncturing a target area with the outer needle and the inner needle, and FIG. 7B is a view for describing a step of causing the inner needle to protrude from the outer needle.

FIG. 8 is a view for describing a step of drawing the outer needle and the inner needle out of the target area and indwelling the implant.

FIG. 9 is a cross-sectional view illustrating a modification of the inner needle illustrated in FIG. 2.

FIG. 10A is a schematic cross-sectional view illustrating an initial state of a lymphangiogenesis inducing device according to a second embodiment, and FIG. 10B is a schematic cross-sectional view illustrating a state where a folding mechanism of the lymphangiogenesis inducing device illustrated in FIG. 10A is unfolded.

### DETAILED DESCRIPTION

### First Embodiment

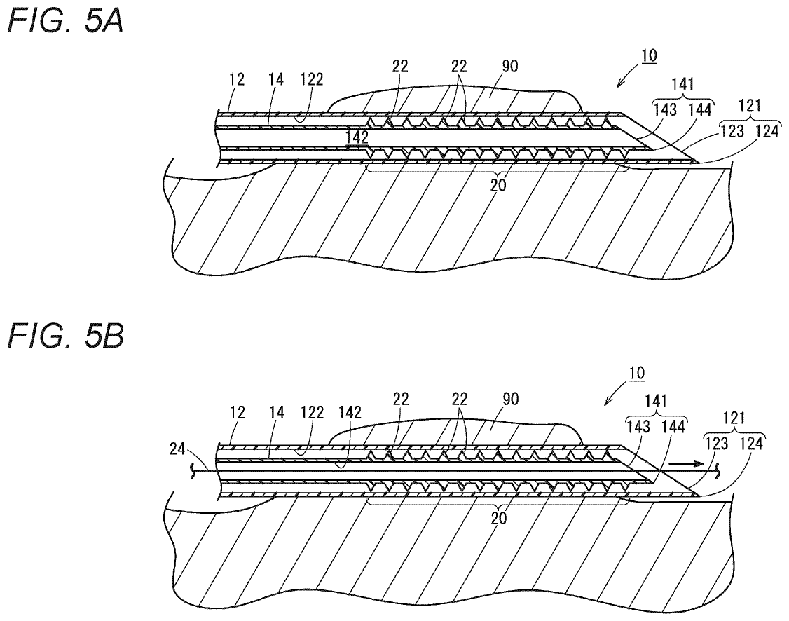

Lymphatic tissue in normal skin and deep tissue (hereinafter also referred to as subcutaneous tissue 90 (see FIG. 5A)) starts from lymphatic capillaries. The lymphatic capillaries have a diameter of 20 to 70 μm. The lymphatic capillaries are distributed in a net-like shape in the dermis just below the epidermis. The lymphatic capillaries lead to pre-collecting lymphatic vessels that are internally located in the dermis. The pre-collecting lymphatic vessels have a diameter of 70 to 150 μm. The pre-collecting lymphatic vessels have a valve structure that allows lymphatic fluid to flow from a distal end toward a proximal end. The pre-collecting lymphatic vessels are distributed in positions deeper than the lymphatic capillaries.

The pre-collecting lymphatic vessels lead to collecting lymphatic vessels. The subcutaneous tissues 90 are abundant in collecting lymphatic vessels. The collecting lymphatic vessels have a diameter of about 0.3 mm in the upper extremity and the trunk and a diameter of about 0.5 mm in the lower extremity. The collecting lymphatic vessels have smooth muscle circumferentially. The collecting lymphatic vessels have a function of guiding lymphatic fluid in the central direction along with automatic movement of the smooth muscle. The collecting lymphatic vessels include shallow collecting ducts in the subcutaneous tissue 90 and deep collecting ducts in tissue deeper than the shallow collecting ducts. The shallow collecting ducts and the deep collecting ducts are connected to lymph nodes on the central side. The collecting lymphatic vessels ultimately lead to veins via lymph nodes.

Lymphedema is said to be caused by dysfunction of a collecting lymphatic vessel. For example, an occluded area generated in a shallow collecting duct may cause lymphedema. The occluded area has a thickened duct wall. The thickened duct wall narrows or occludes a channel of a collecting lymphatic vessel. Stenosis or occlusion of a collecting lymphatic vessel inhibits drainage of tissue fluid from a lymphatic vessel, which develops into lymphedema and causes swelling of the extremities.

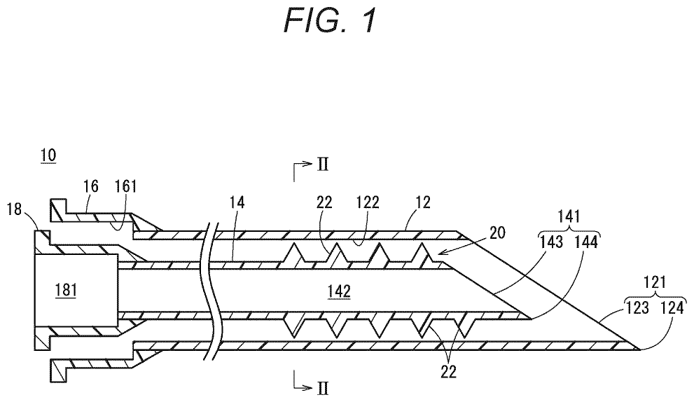

A lymphangiogenesis inducing device 10 of this embodiment illustrated in FIG. 1 is used for treatment of lymphatic vessel tissue having the above dysfunction. The lymphangiogenesis inducing device 10 is used to form a new lymphatic vessel that is to be a bypass of an occluded area.

As illustrated in the drawing, the lymphangiogenesis inducing device 10 includes an outer needle 12, an inner needle 14, an outer needle hub 16, and an inner needle hub 18. The outer needle 12 has a long tubular shape. The outer needle hub 16 is connected to a proximal portion of the outer needle 12. The outer needle hub 16 has a shape that is easy to grip and is used for operating the outer needle 12. An operator such as a doctor holds the outer needle hub 16 to operate the outer needle 12.

The outer needle 12 also includes a needle point 121 and a through-hole 122. The needle point 121 is formed on a distal end of the outer needle 12. The needle point 121 has a bevel 123 cut obliquely relative to the extending direction of the outer needle 12. The bevel 123 has a distal end provided with a sharp tip 124 capable of puncturing the subcutaneous tissue 90.

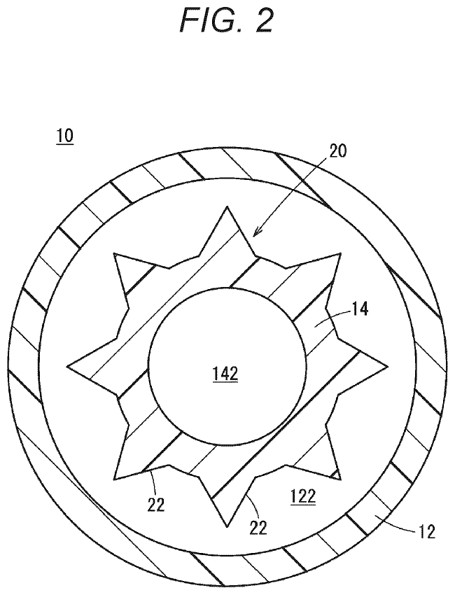

The through-hole 122 is formed inside the outer needle 12. The through-hole 122 extends along the central axis of the outer needle 12 and penetrates the outer needle 12 in the axial direction from the distal end to proximal end. The through-hole 122 has a distal end opened at the bevel 123. The proximal end of the through-hole 122 opens at the inside of the outer needle hub 16. The inside of the outer needle hub 16 is provided with a hollow portion 161 that communicates with the through-hole 122. As illustrated in FIG. 2, the outer needle 12 and the through-hole 122 are formed to have a circular cross section. Note that the cross sections of the outer needle 12 and the through-hole 122 are not limited to a circular shape, and may have a rectangular shape or a polygonal shape.

The outer needle 12 is formed of a metal material such as stainless steel, aluminum, aluminum alloys, titanium, or titanium alloys (for example, nickel-titanium alloy). The material of the outer needle 12 may be hard resin or ceramics and so on. The outer needle 12 has an outside diameter of, for example, about 0.5 to 4.0 mm, and the through-hole 122 has a diameter of, for example, about 0.25 to 3.5 mm.

As illustrated in FIG. 1, the inner needle 14 is inserted into the through-hole 122 of the outer needle 12. The inner needle 14 is a long tubular member having a length equivalent to or longer than the entire length of the outer needle 12. The inner needle 14 has a proximal portion protruding to the proximal side of the outer needle 12. The inner needle hub 18 is fixed to the proximal portion of the inner needle 14. The inner needle hub 18 has an outer shape larger than that of the inner needle 14. An operator grips the inner needle hub 18 to move the inner needle 14 forward or backward in the axial direction.

Around a distal end of the inner needle 14, there is a part provided with a wound imparting structure 20. In this embodiment, the wound imparting structure 20 is formed on a part of the inner needle 14 on the distal side. The wound imparting structure 20 includes a plurality of protrusions 22 protruding outward from the inner needle 14. As illustrated in FIG. 2, the protrusions 22 are formed over the entire region of the inner needle 14 in the circumferential direction. The protrusions 22 are separated from each other in the circumferential direction and the axial direction. Each protrusion 22 has a sharp distal end. The distal ends of the protrusions 22 have sharpness (radius of curvature) to such an extent as to cause minor wounds on the subcutaneous tissue 90. The protrusions 22 are joined to the outer periphery of the inner needle 14 by welding, brazing, adhesion, plating, or the like. The protrusions 22 may be formed in an integrated manner with the inner needle 14 by cutting out outer peripheral portions of the inner needle 14. As illustrated in FIG. 1, among the entire length of the inner needle 14, the wound imparting structure 20 may be formed at least in a range where the wound imparting structure 20 can protrude from the needle point 121 of the outer needle 12. Alternatively, the wound imparting structure 20 may be formed over the entire length of the inner needle 14.

The protrusions 22 are randomly arranged on the outer peripheral portions of the inner needle 14. Note that the protrusions 22 are not particularly limited in arrangement. The protrusions 22 may be arranged on the outer peripheral portions of the inner needle 14 in a helical manner. Alternatively, the protrusions 22 may be arranged on the outer peripheral portions of the inner needle 14 in a circular manner. The protrusions 22 have a protruding height of, for example, but not particularly limited to, 0.1 to 0.5 mm from the outer periphery of the inner needle 14.

The wound imparting structure 20 has an outside diameter smaller than the diameter of the through-hole 122 of the outer needle 12. This configuration enables the inner needle 14 including the wound imparting structure 20 to move forward or backward smoothly through the through-hole 122 of the outer needle 12. Collecting lymphatic vessels extend along nerve bundles or blood vessels. Therefore, if the protrusions 22 of the wound imparting structure 20 have too large a radial dimension, there is a risk of damaging peripheral nerve bundles and blood vessels. In order to prevent such a situation, the wound imparting structure 20 (the inner needle 14 including the protrusions 22) preferably has an outside diameter of 6.0 mm or less.

The inner needle 14 further includes a needle point 141 and a lumen 142 (cavity portion). The needle point 141 has a bevel 143 inclined relative to the axial direction of the inner needle 14. The bevel 143 has a distal end provided with a sharp tip 144. Note that the needle point 141 of the inner needle 14 need not be sharp. In other words, the needle point 141 of the inner needle 14 may be a blunt needle point having an end face perpendicular to the axial direction and a smooth curved surface obtained by chamfering the edge of the end face.

The lumen 142 of the inner needle 14 extends along the axial direction of the inner needle 14 and penetrates the inner needle 14 from the distal end to proximal end. The lumen 142 has a distal end opened at the needle point 141 and a proximal end opened toward the inner needle hub 18. The inner needle hub 18 has a hollow portion 181 that communicates with the lumen 142.

The inner needle 14 excluding the wound imparting structure 20 illustrated in FIG. 1 may have a diameter of about 0.2 to 3.0 mm. The lumen 142 of the inner needle 14 may have an inside diameter of about 0.1 to 2.8 mm. The inner needle 14 including the protrusions 22 has strength capable of puncturing the body and has hardness and toughness capable of withstanding abrasion with the subcutaneous tissue 90. Examples of materials for the inner needle 14 and the wound imparting structure 20 (protrusions 22) include metal materials such as stainless steel, aluminum, aluminum alloys, titanium, titanium alloys (for example, nickel-titanium alloy), hard resin, and ceramics. The protrusions 22 may employ a material having higher hardness than that of the inner needle 14.

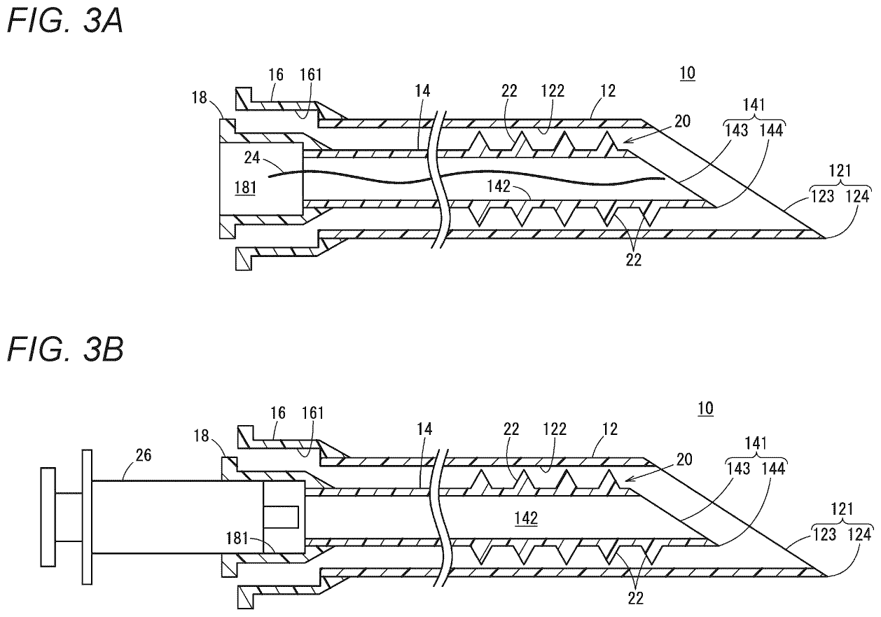

As illustrated in FIG. 3A, it is possible to insert an implant 24 into the lumen 142 of the inner needle 14 through the hollow portion 181 of the inner needle hub 18. The implant 24 is a long biodegradable polymer. When the implant 24 is indwelled in the subcutaneous tissue 90, cells that induce lymphangiogenesis are fixed to the implant 24, thereby promoting lymphangiogenesis. Examples of a material for the implant 24 include porous collagen fibers. Porous collagen fibers have good compatibility with the subcutaneous tissue 90 and are excellent in cell fixability due to their porosity.

In addition, as illustrated in FIG. 3B, the lymphangiogenesis inducing device 10 enables a syringe 26 to connect to the inner needle hub 18. The syringe 26 contains a drug solution. When the syringe 26 is handled by an operator, the drug solution is injected into the subcutaneous tissue 90 through the lumen 142.

The lymphangiogenesis inducing device 10 of this embodiment is configured as described above. Described below is a procedure using the lymphangiogenesis inducing device 10.

Prior to a procedure, an operator determines an occluded area of a lymphatic vessel in advance. The occluded area of the lymphatic vessel can be determined by a method such as ICG fluorescence lymphangiography, lymphoscintigraphy, MRI, CT, and diagnostic ultrasound imaging. Next, the operator determines a route for lymphangiogenesis. For example, the operator identifies the occluded area of the lymphatic vessel and decides a route leading to a non-occluded lymphatic vessel adjacent to the occluded area. In addition, for example, the operator decides a route that bypasses the occluded area of the lymphatic vessel.

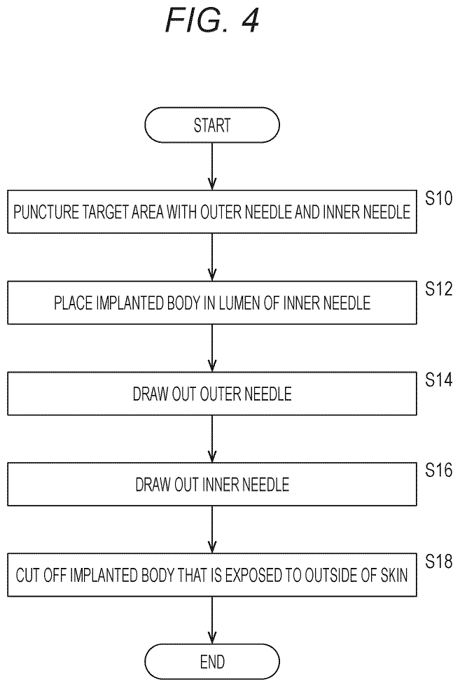

Next, as illustrated in steps S10 to S18 of FIG. 4, a procedure using the lymphangiogenesis inducing device 10 is performed. First, as illustrated in step S10, the operator punctures a target area with the lymphangiogenesis inducing device 10. As illustrated in FIG. 5A, in a first procedure, the outer needle 12 is caused to penetrate through the subcutaneous tissue 90. The puncturing with the outer needle 12 is performed in a state where the inner needle 14 is inserted into the through-hole 122 so as to prevent the subcutaneous tissue 90 from entering and blocking the through-hole 122 of the outer needle 12.

Next, as illustrated in step S12 of FIG. 4 and FIG. 5B, the operator places the implant 24 in the lumen 142 of the inner needle 14. The implant 24 is inserted to protrude toward the distal side from the lumen 142 of the inner needle 14 and the through-hole 122 of the outer needle 12. Accordingly, a part near the distal end and a part near the proximal end of the implant 24 are not exposed from the subcutaneous tissue 90. Note that the implant 24 may be placed in the inner needle 14 in advance. In this case, step S12 may be omitted.

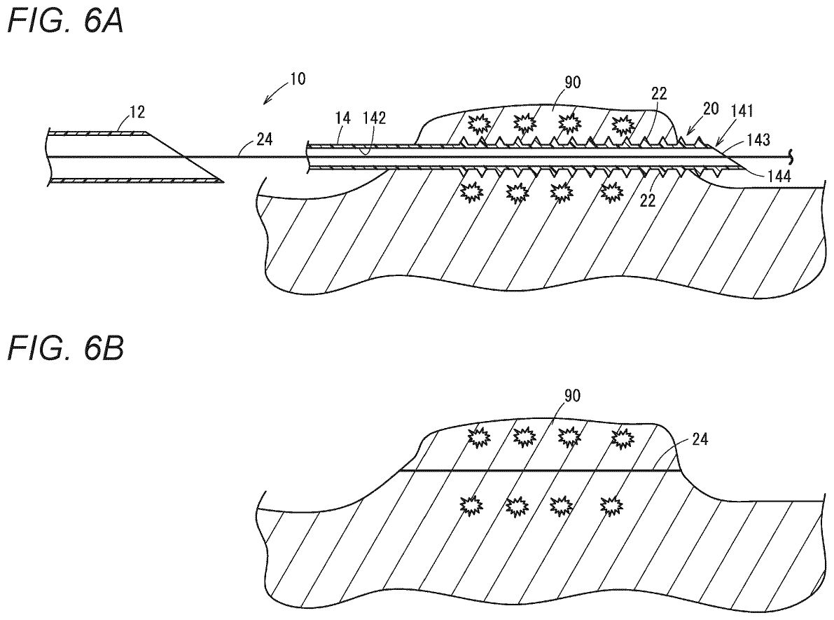

Next, as illustrated in step S14 of FIG. 4 and FIG. 6A, the operator draws the outer needle 12 out of the subcutaneous tissue 90. When the outer needle 12 is drawn out, the wound imparting structure 20 of the inner needle 14 is exposed and comes into contact with the subcutaneous tissue 90.

Then, as illustrated in step S16 of FIG. 4, the operator draws the inner needle 14 out of the subcutaneous tissue 90. When the inner needle 14 is drawn out, the protrusions 22 rub against the subcutaneous tissue 90 and form many minor wounds on the subcutaneous tissue 90. The minor wounds are formed along a route through which the inner needle 14 is drawn. To form wounds more reliably, the operator draws out the inner needle 14 while adding forward movement of the inner needle 14 and rotational movement of the inner needle 14. The inner needle 14 is drawn out but the implant 24 is left in the subcutaneous tissue 90. Through this step S16, it is possible to form wounds in the subcutaneous tissue 90, and simultaneously, it is possible to indwell the implant 24 in the subcutaneous tissue 90. A part of the distal end and a part of the proximal end of the implant 24 are exposed from the subcutaneous tissue 90.

Next, as illustrated in step S18 of FIG. 4, the operator cuts off the implant 24 that is exposed from the subcutaneous tissue 90. Through this step, as illustrated in FIG. 6B, the implantation of the implant 24 into the subcutaneous tissue 90 is completed.

In this manner, the first procedure is completed. The operator repeats the operations of steps S10 to S18 for other routes, thereby completing the formation of wounds and the indwelling of the implant 24 for all desired routes.

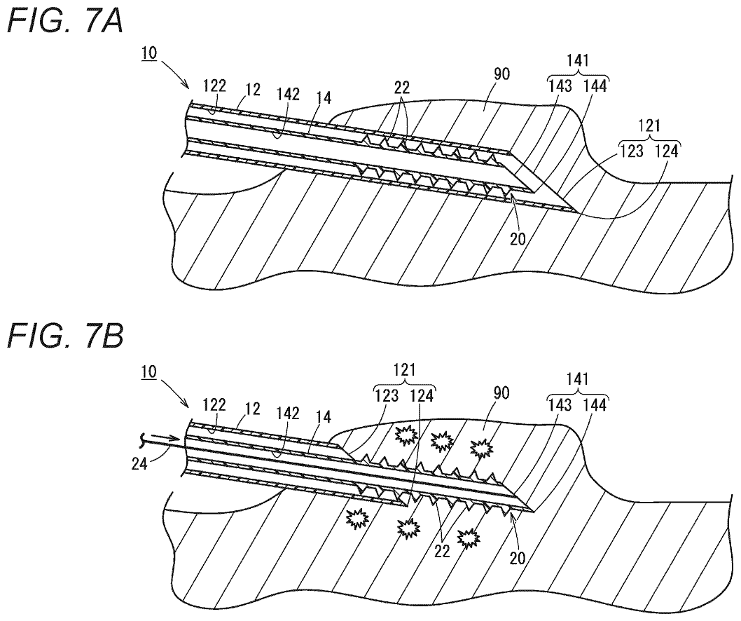

Next, a second procedure will be described. As illustrated in FIG. 7A, in the second procedure, the operator punctures the subcutaneous tissue 90 with the lymphangiogenesis inducing device 10 in such a manner that the lymphangiogenesis inducing device 10 does not penetrate entirely through the subcutaneous tissue 90. A predetermined area of the subcutaneous tissue 90 is punctured with the needle point 121 of the outer needle 12. Next, as illustrated in FIG. 7B, the operator places the implant 24 in the lumen 142 of the inner needle 14. After that, the operator moves the outer needle 12 backward toward the proximal side or causes the inner needle 14 to protrude toward the distal side from the needle point 121 of the outer needle 12. Accordingly, the wound imparting structure 20 of the inner needle 14 is exposed, and wounds are formed in the subcutaneous tissue 90. The operator reliably forms wounds in the subcutaneous tissue 90 by moving the inner needle 14 forward or backward a plurality of times.

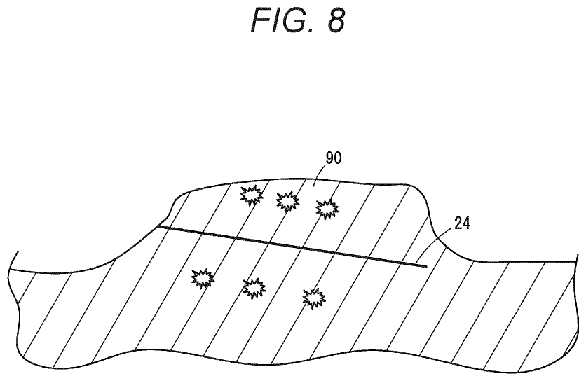

After that, as illustrated in FIG. 8, the operator draws out the outer needle 12 and the inner needle 14 sequentially. Finally, a proximal portion of the implant 24 exposed from the subcutaneous tissue 90 is cut off to complete the procedure. Note that, in the second procedure, in a state of FIG. 7A or 7B, the drug solution may be injected into the subcutaneous tissue 90 through the inner needle 14.

According to the procedure, during healing of linear wounds illustrated in FIG. 8, immune cells accumulate in the wounds due to immune responses to the wounds. Various cells accumulate around the wounds, and some cells fix to the implant 24. The cells fixed to the implant 24 and the immune cells accumulated in the wounds enhance the expression of lymphatic growth factors. Accordingly, the lymphangiogenesis inducing device 10 of this embodiment can induce lymphangiogenesis.

Lymphangiogenesis emerges and proceeds from an existing lymphatic vessel (see Lymphangiogenesis: Molecular mechanisms and future promise). Therefore, a shallow collecting duct and a deep collecting duct are regenerated by extending from an existing lymphatic vessel (not illustrated in FIG. 8). In this manner, the lymphangiogenesis inducing device 10 of this embodiment enables regeneration of a lymphatic vessel that bypasses an occluded area or a lymphatic vessel that connects adjacent existing lymphatic vessels.

### First Modification of First Embodiment

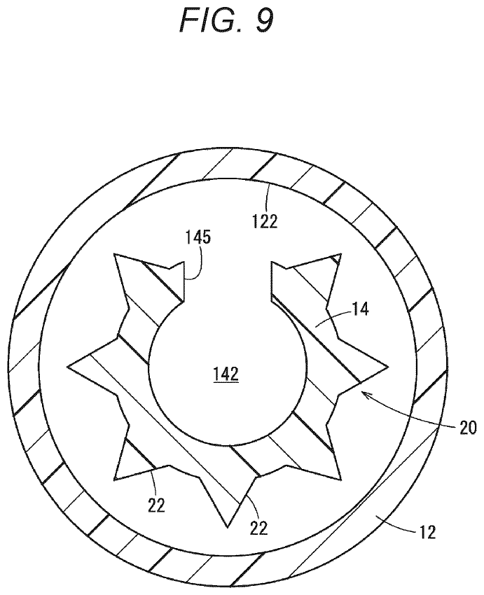

As illustrated in FIG. 9, in this modification, the shape of the lumen 142 (cavity portion) of the inner needle 14 is modified. The inner needle 14 of this modification has a cutout groove 145 obtained by cutting out a peripheral wall of the inner needle 14 in part of the lumen 142. The lumen 142 is exposed through the cutout groove 145. The cutout groove 145 like a trench extends in the axial direction of the inner needle 14. According to this modification, it is possible to obtain an effect similar to that of the lymphangiogenesis inducing device 10 illustrated in FIG. 1. Note that the cavity portion of the inner needle 14 is not limited to the lumen 142 and may be a groove having a V-shaped cross section or a groove having a rectangular cross section.

### Second Embodiment

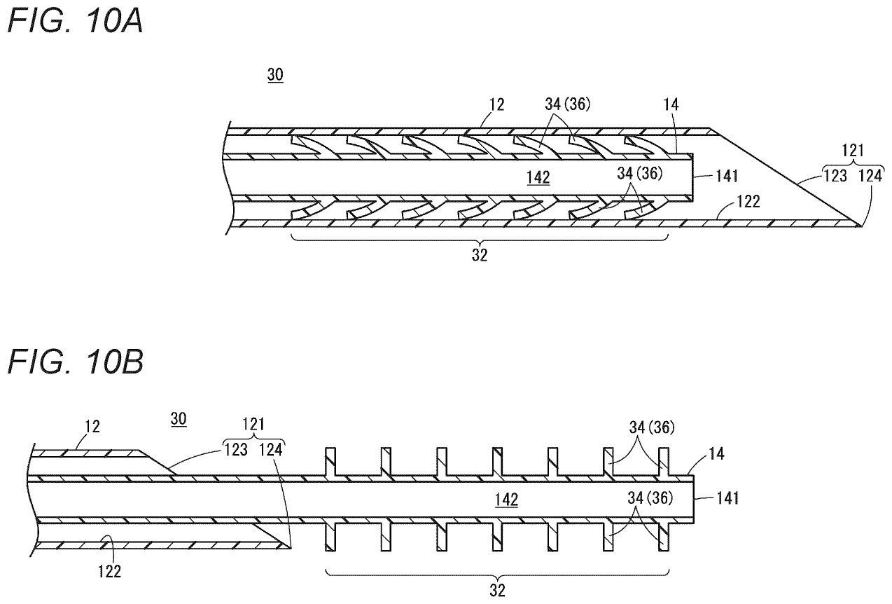

As illustrated in FIG. 10A, a lymphangiogenesis inducing device 30 of this embodiment has a wound imparting structure 32 different from that of the lymphangiogenesis inducing device 10 described with reference to FIG. 1. In the lymphangiogenesis inducing device 30, the same components as those of the lymphangiogenesis inducing device 10 illustrated in FIG. 1 are denoted by the same reference numerals, and details thereof will be omitted.

The wound imparting structure 32 of this embodiment includes a plurality of rod-like protrusions 34. The protrusions 34 are longer than the protrusions 22 illustrated in FIG. 1. Proximal ends of the protrusions 34 are joined to an inner needle 14. Each protrusion 34 has a folding mechanism 36 that bends and folds with respect to the inner needle 14. The folding mechanism 36 is formed by each elastically deformable protrusion 34 itself. The folding mechanism 36 is not limited to the configuration and may include a hinge connected to the proximal end of each protrusion 34.

The protrusions 34 are folded while the wound imparting structure 32 is housed in a through-hole 122 of an outer needle 12. With this configuration, the inner needle 14 including the protrusions 34 can smoothly move forward or backward inside the outer needle 12.

When the wound imparting structure 32 of the inner needle 14 is protruded from a distal end of the outer needle 12, the protrusions 34 erect by an elastic restoring force. Accordingly, as illustrated in FIG. 10B, the protrusions 34 become unfolded. In this manner, the wound imparting structure 32 increases a range for the protrusions 34 to protrude. In a state where the protrusions 34 are unfolded, the protruding height of each protrusion 34 from the inner needle 14 is about 0.2 to 1.0 mm on average.

Therefore, in the inner needle 14 of this embodiment, the outside diameter of the wound imparting structure 32 is larger than the diameter of the through-hole 122 of the outer needle 12. The protrusions 34 maintain erected even when the inner needle 14 is drawn toward the proximal side in a subcutaneous tissue 90.

Even the lymphangiogenesis inducing device 30 of this embodiment enables wounding in the subcutaneous tissue 90 and indwelling of an implant 24.

Note that the present invention is not limited to the described embodiments, and various configurations can be adopted without departing from the gist of the present invention.

1. A lymphangiogenesis inducing device comprising:
an outer needle comprising a needle point insertable into a subcutaneous tissue, and a through-hole extending in an axial direction and penetrating the outer needle; and
an inner needle inserted into the through-hole, wherein:
the inner needle comprises a cavity portion extending along the axial direction, and a wound imparting structure that is formed in a part of a distal end configured to protrude past the needle point of the outer needle and configured to cause a wound on the subcutaneous tissue, and
the wound imparting structure comprises one or more protrusions protruding outward from the inner needle.

2. The lymphangiogenesis inducing device according to claim 1, wherein:
the one or more protrusions comprise a plurality of protrusions protruding radially outward from a proximal end of the inner needle.

3. The lymphangiogenesis inducing device according to claim 1, wherein:
each of the one or more protrusions comprises a folding mechanism configured to allow the protrusion to be folded when inserted into the through-hole and unfolded outward when protruding past the needle point of the outer needle.

4. The lymphangiogenesis inducing device according to claim 1, further comprising:
an inner needle hub located at a proximal end of the inner needle, the inner needle being configured to protrude or retract relative to the needle point of the outer needle when the inner needle hub is operated.

5. The lymphangiogenesis inducing device according to claim 4, wherein:
the inner needle hub comprises a hollow portion communicating with the cavity portion of the inner needle, and allows a drug solution or an implant to be supplied to the cavity portion of the inner needle via the hollow portion.

6. The lymphangiogenesis inducing device according to claim 5, wherein:
the implant is a porous and filamentous biodegradable polymer.

7. The lymphangiogenesis inducing device according to claim 5, wherein:
the implant is configured to be indwelled in the subcutaneous tissue after the outer needle and the inner needle are drawn out of the subcutaneous tissue.

8. The lymphangiogenesis inducing device according to claim 1, wherein:
the lymphangiogenesis inducing device further comprises an implant located in the cavity portion of the inner needle.

9. A method for inducing lymphangiogenesis to treat an occluded area of a lymphatic vessel, the method comprising:
providing a lymphangiogenesis inducing device comprising:
an outer needle comprising a needle point insertable into a subcutaneous tissue, and a through-hole extending in an axial direction and penetrating the outer needle, and
an inner needle inserted into the through-hole, wherein:
the inner needle comprises a cavity portion extending along the axial direction, and a wound imparting structure that is formed in a part of a distal end configured to protrude past the needle point of the outer needle and configured to cause a wound on the subcutaneous tissue, and
the wound imparting structure comprises one or more protrusions protruding outward from the inner needle;
puncturing the subcutaneous tissue with the lymphangiogenesis inducing device while the inner needle is located in the through-hole;
placing an implant in the cavity portion;
drawing the outer needle proximally or pushing the inner needle distally, such that the wound imparting structure contacts the subcutaneous tissue;
drawing the inner needle out of the subcutaneous tissues so as to cause wounds on the subcutaneous tissue, such that the implant is indwelled in the subcutaneous tissue.

10. A method for inducing lymphangiogenesis to treat an occluded area of a lymphatic vessel, the method comprising:
providing a lymphangiogenesis inducing device comprising:
an outer needle comprising a needle point insertable into a subcutaneous tissue, and a through-hole extending in an axial direction and penetrating the outer needle, and
an inner needle inserted into the through-hole, and
an implant located in the cavity portion of the inner needle, wherein:
the inner needle comprises a cavity portion extending along the axial direction, and a wound imparting structure that is formed in a part of a distal end configured to protrude past the needle point of the outer needle and configured to cause a wound on the subcutaneous tissue, and
the wound imparting structure comprises one or more protrusions protruding outward from the inner needle;
puncturing the subcutaneous tissue with the lymphangiogenesis inducing device while the inner needle is located in the through-hole;
drawing the outer needle proximally or pushing the inner needle distally, such that the wound imparting structure contacts the subcutaneous tissue;
drawing the inner needle out of the subcutaneous tissues so as to cause wounds on the subcutaneous tissue, such that the implant is indwelled in the subcutaneous tissue.
